(19) United States
(12) Patent Application Publication
(10) Pub. No.: US 2023/0309853 A1
(43) Pub. Date: Oct. 5, 2023

(54) NOISE IN ELECTRO-ANATOMIC SIGNALS

(71) Applicant: BIOSENSE WEBSTER (ISRAEL) LTD., Yokneam (IL)

(72) Inventors: Stanislav Chisty, Yokneam (IL); Ran Sharon, Petah Tikva (IL); Niv Derech, Sdot Yam (IL); Aviram Weiss Mizrahi, Tiberias (IL); Ariel Leonardo Noyjovich, Harish (IL)

(21) Appl. No.: 17/709,623

(22) Filed: Mar. 31, 2022

(51) Int. Cl. A61B 5/06; A61B 34/20

(52) U.S. Cl. CPC A61B 5/063; A61B 34/20; A61B 5/068; A61B 2034/2051; A61B 2034/2072

(57) ABSTRACT

In one exemplary mode, a medical system includes a catheter configured to be inserted into a body part of a living subject, and comprising multiple electrodes configured to contact tissue of the body part, a display, and processing circuitry configured to receive a signal from one of the electrodes, find a noise measurement of the signal, and render to the display a dynamic indication of the noise measurement.

### FIELD OF THE DISCLOSURE

The present disclosure relates to medical systems, and in particular, but not exclusively to, catheter-based systems.

### BACKGROUND

A wide range of medical procedures involve placing probes, such as catheters, within a patient's body. Location sensing systems have been developed for tracking such probes. Magnetic location sensing is one of the methods known in the art. In magnetic location sensing, magnetic field generators are typically placed at known locations external to the patient. A magnetic field sensor within the distal end of the probe generates electrical signals in response to these magnetic fields, which are processed to determine the coordinate locations of the distal end of the probe. These methods and systems are described in U.S. Pat. Nos. 5,391,199, 6,690,963, 6,484,118, 6,239,724, 6,618,612 and 6,332,089, in PCT International Publication No. WO 1996/005768, and in U.S. Patent Application Publications Nos. 2002/0065455 and 2003/0120150 and 2004/0068178, whose disclosures are all incorporated herein by reference. Locations may also be tracked using impedance or current based systems.

One medical procedure in which these types of probes or catheters have proved extremely useful is in the treatment of cardiac arrhythmias. Cardiac arrhythmias and atrial fibrillation in particular, persist as common and dangerous medical ailments, especially in the aging population.

Diagnosis and treatment of cardiac arrhythmias include mapping the electrical properties of heart tissue, especially the endocardium and the heart volume, and selectively ablating cardiac tissue by application of energy. Such ablation can cease or modify the propagation of unwanted electrical signals from one portion of the heart to another. The ablation process destroys the unwanted electrical pathways by formation of non-conducting lesions. Various energy delivery modalities have been disclosed for forming lesions, and include use of microwave, laser and more commonly, radiofrequency energies to create conduction blocks along the cardiac tissue wall. In a two-step procedure, mapping followed by ablation, electrical activity at points within the heart is typically sensed and measured by advancing a catheter containing one or more electrical sensors into the heart, and acquiring data at a multiplicity of points. These data are then utilized to select the endocardial target areas at which the ablation is to be performed.

### BRIEF DESCRIPTION OF THE DRAWINGS

The present disclosure will be understood from the following detailed description, taken in conjunction with the drawings in which:

FIG. 1 is a schematic view of a medical procedure system constructed and operative in accordance with an exemplary mode of the present disclosure;

FIG. 2 is a schematic view of a catheter for use in the system of FIG. 1;

FIG. 3 is a flowchart including steps in a method of noise level presentation for use in the system of FIG. 1;

FIG. 4 is a schematic view of a noise level presentation in the system of FIG. 1;

FIG. 5 is a flowchart including steps in a method of setting a noise level for selecting signals in the system of FIG. 1;

FIG. 6 is a schematic view of a user interface screen used to set a noise level for selecting signals in the system of FIG. 1; and

FIG. 7 is a schematic view of an electro-anatomical map generated by the system of FIG. 1.

### DESCRIPTION OF EXAMPLES

### Overview

Noise is added to intracardiac electrogram (IEGM) signals (captured by catheters) and electrocardiogram (ECG) signals (captured by body surface patches) in an electrophysiological (EP) laboratory from equipment in the EP laboratory and other sources such as signal processing circuits, the catheter, and a position (e.g., magnetic) tracking system. For example, as cardiac signals are carried in wires and cables from a catheter and/or body-surface electrodes, the cardiac signals pick up noise generated in the EP laboratory. Each EP laboratory may have its own noise profile based on the equipment operating in the EP laboratory. The noise distorts the IEGM and/or ECG signals and may prevent useful analysis and use of the signals.

Physicians generally do not want to use electrodes which are picking up too much noise for mapping. Electrodes may be selected by carefully examining the intracardiac signals (IEGMs) captured by the electrodes to determine if the electrodes are picking up too much noise. However, this is very time consuming, especially for catheters with tens of electrodes. Additionally, the amount of noise being picked up by an electrode changes over time based on its position and other factors.

Exemplary modes of the present disclosure solve at least some of the above problems by finding noise measurements for respective signals captured by electrodes, selecting signals with noise measurements below a given noise level, and rendering to a display electro-anatomical data (e.g., IEGM and/or ECG traces, and/or an electro-anatomical map) based on the selected signals while more noisy signals are not used to generate the displayed electro-anatomical data. For example, an electro-anatomical map may be generated from the selected signals and then rendered to the display.

In some exemplary modes, a user interface is provided to receive a user input of a noise level used to select the signals. In some exemplary modes, the user interface includes a noise level selector slider to enable a user to select the given noise level by moving the slider to select the desired noise level. In some exemplary modes, the user interface may include other mapping option selectors such as cycle length, pattern matching, position stability, and local activation time (LAT) stability.

The noise measurements may be computed as a function of the magnitudes of frequencies associated with noise. For example, high frequency components of the signals captured by the catheter are probably of non-electro-anatomic origin. For example, frequencies above 70 or 150 Hz (depending upon the arrythmia) are probably of non-electro-anatomic origin. The noise measurements may be based on an absolute measure of the noise, or as a type of signal to noise ratio.

In some exemplary modes, dynamic indications of the noise measurements (that change is real-time) of each the signals are rendered to a display so that the physician can easily see the noise level of each of the signals. In some exemplary modes, the dynamic indications are graphical representations such as colored indicators which change color responsively to the respective levels of noise measurements of the signals. The colored indicator may be colored lines. For example, “green” for no noise, “yellow” for low noise, “orange” for medium noise, and “red” for high noise.

In some exemplary modes, the catheter may include multiple splines with electrodes placed along the splines. The graphical representations (e.g., colored lines) maybe grouped according to the splines. For example, five colored vertical lines may be grouped together for five electrodes of one of the splines of a multi-spline mapping catheter. The display may include eight groups (labeled A-H) of five lines corresponding to the five electrodes of each of the eight splines (labeled A-H) of the mapping Catheter.

### System Description

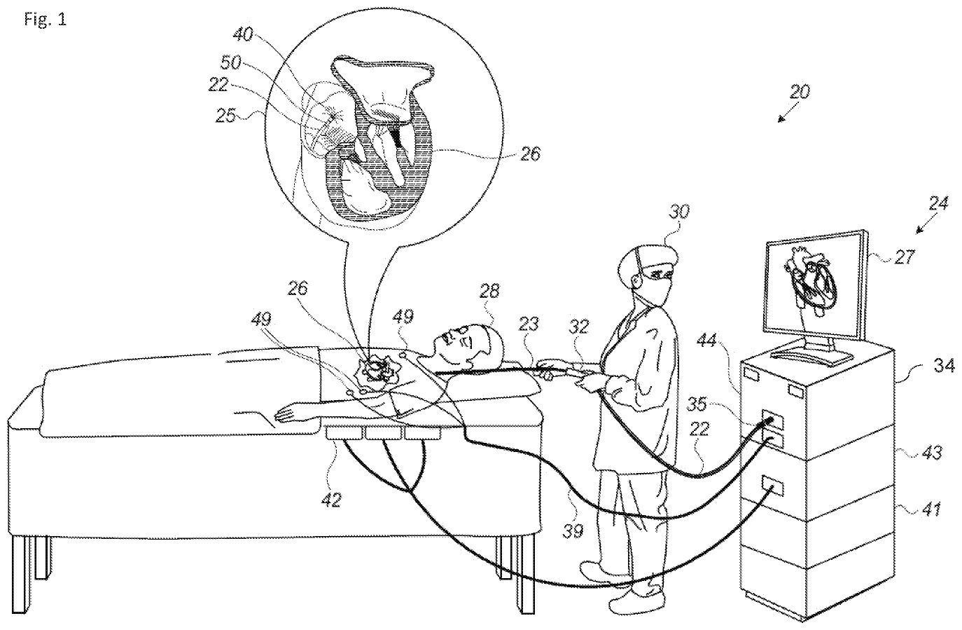

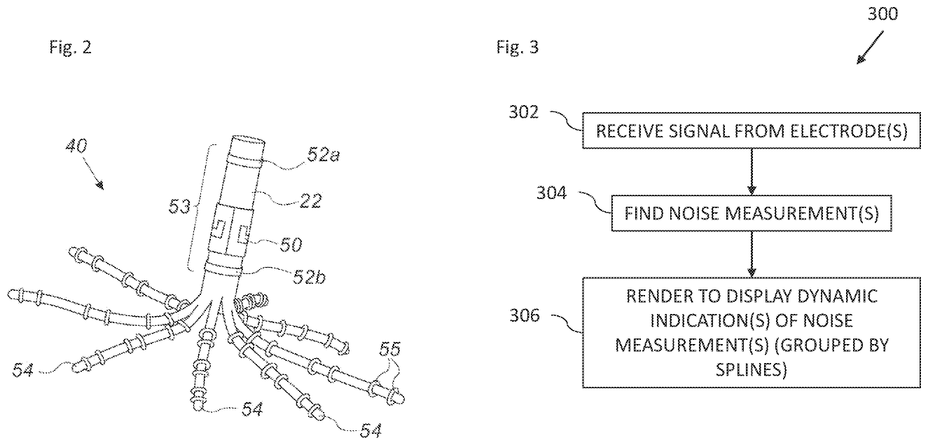

Reference is now made to FIG. 1, which is a schematic view of a medical procedure system 20 constructed and operative in accordance with an exemplary mode of the present disclosure. Reference is also made to FIG. 2, which is a schematic view of a catheter 40 for use in the system 20 of FIG. 1.

The medical procedure system 20 is used to determine the position of the catheter 40, seen in an inset 25 of FIG. 1 and in more detail in FIG. 2. The catheter 40 includes a shaft 22 and a plurality of deflectable arms 54 (only some labeled for the sake of simplicity) for inserting into a body-part of a living subject. The deflectable arms 54 have respective proximal ends connected to the distal end of the shaft 22.

The catheter 40 includes a position sensor 53 disposed on the shaft 22 in a predefined spatial relation to the proximal ends of the deflectable arms 54. The position sensor 53 may include a magnetic sensor 50 and/or at least one shaft electrode 52. The magnetic sensor 50 may include at least one coil, for example, but not limited to, a dual-axis or a triple axis coil arrangement to provide position data for location and orientation including roll. The catheter 40 includes multiple electrodes 55 (only some are labeled in FIG. 2 for the sake of simplicity) disposed at different, respective locations along each of the deflectable arms 54. The electrodes 55 are configured to contact tissue of the body part. Typically, the catheter 40 may be used for mapping electrical activity in a heart of the living subject using the electrodes 55, or for performing any other suitable function in a body-part of a living subject.

The medical procedure system 20 may determine a position and orientation of the shaft 22 of the catheter 40 based on signals provided by the magnetic sensor 50 and/or the shaft electrodes 52 (proximal electrode 52a and distal electrode 52b) fitted on the shaft 22, on either side of the magnetic sensor 50. The proximal electrode 52a, the distal electrode 52b, the magnetic sensor 50 and at least some of the electrodes 55 are connected by wires running through the shaft 22 via a catheter connector 35 to various driver circuitries in a console 24. In some exemplary modes, at least two of the electrodes 55 of each of the deflectable arms 54, the shaft electrodes 52, and the magnetic sensor 50 are connected to the driver circuitries in the console 24 via the catheter connector 35. In some exemplary modes, the distal electrode 52b and/or the proximal electrode 52a may be omitted.

The illustration shown in FIG. 2 is chosen purely for the sake of conceptual clarity. Other configurations of shaft electrodes 52 and electrodes 55 are possible. Additional functionalities may be included in the position sensor 53. Elements which are not relevant to the disclosed exemplary modes of the disclosure, such as irrigation ports, are omitted for the sake of clarity.

A physician 30 navigates the catheter 40 to a target location in a body part (e.g., a heart 26) of a patient 28 by manipulating the shaft 22 using a manipulator 32 near the proximal end of the catheter 40 and/or deflection from a sheath 23. The catheter 40 is inserted through the sheath 23, with the deflectable arms 54 gathered together, and only after the catheter 40 is retracted from the sheath 23, the deflectable arms 54 are able to spread and regain their intended functional shape. By containing deflectable arms 54 together, the sheath 23 also serves to minimize vascular trauma on its way to the target location.

Console 24 comprises processing circuitry 41, typically a general-purpose computer and a suitable front end and interface circuits 44 for generating signals in, and/or receiving signals from, body surface electrodes 49 which are attached by wires running through a cable 39 to the chest and to the back, or any other suitable skin surface, of the patient 28.

Console 24 further comprises a magnetic-sensing sub-system. The patient 28 is placed in a magnetic field generated by a pad containing at least one magnetic field radiator 42, which is driven by a unit 43 disposed in the console 24. The magnetic field radiator(s) 42 is configured to transmit alternating magnetic fields into a region where the body-part (e.g., the heart 26) is located. The magnetic fields generated by the magnetic field radiator(s) 42 generate direction signals in the magnetic sensor 50. The magnetic sensor 50 is configured to detect at least part of the transmitted alternating magnetic fields and provide the direction signals as corresponding electrical inputs to the processing circuitry 41.

In some exemplary modes, the processing circuitry 41 uses the position-signals received from the shaft electrodes 52, the magnetic sensor 50 and the electrodes 55 to estimate a position of the catheter 40 inside an organ, such as inside a cardiac chamber. In some exemplary modes, the processing circuitry 41 correlates the position signals received from the electrodes 52, 55 with previously acquired magnetic location-calibrated position signals, to estimate the position of the catheter 40 inside a cardiac chamber. The position coordinates of the shaft electrodes 52 and the electrodes 55 may be determined by the processing circuitry 41 based on, among other inputs, measured impedances, or on proportions of currents distribution, between the electrodes 52, 55 and the body surface electrodes 49. The console 24 drives a display 27, which shows the distal end of the catheter 40 inside the heart 26.

The method of position sensing using current distribution measurements and/or external magnetic fields is implemented in various medical applications, for example, in the Carto® system, produced by Biosense Webster Inc. (Irvine, California), and is described in detail in U.S. Pat. Nos. 5,391,199, 6,690,963, 6,484,118, 6,239,724, 6,618,612, 6,332,089, 7,756,576, 7,869,865, and 7,848,787, in PCT Patent Publication WO 96/05768, and in U.S. Patent Application Publication Nos. 2002/0065455 A1, 2003/0120150 A1 and 2004/0068178 A1.

The Carto@3 system applies an Active Current Location (ACL) impedance-based position-tracking method. In some exemplary modes, using the ACL method, the processing circuitry 41 is configured to create a mapping (e.g., current-position matrix (CPM)) between indications of electrical impedance and positions in a magnetic coordinate frame of the magnetic field radiator(s) 42. The processing circuitry 41 estimates the positions of the shaft electrodes 52 and the electrodes 55 by performing a lookup in the CPM.

Processing circuitry 41 is typically programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

FIG. 1 shows only elements related to the disclosed techniques, for the sake of simplicity and clarity. The system 20 typically comprises additional modules and elements that are not directly related to the disclosed techniques, and thus are intentionally omitted from FIG. 1 and from the corresponding description.

The catheter 40 described above includes eight deflectable arms 54. Any suitable catheter may be used instead of the catheter 40, for example, a catheter with a different number of flexible arms and/or electrodes per arm, or a different probe shape such as a balloon catheter or a lasso catheter, by way of example only.

The medical procedure system 20 may also perform ablation of heart tissue using any suitable catheter, for example using the catheter 40 or a different catheter and any suitable ablation method. The console 24 may include an RF signal generator 34 configured to generate RF power to be applied by an electrode or electrodes of a catheter connected to the console 24, and one or more of the body surface electrodes 49, to ablate a myocardium of the heart 26. The console 24 may include a pump (not shown), which pumps irrigation fluid into an irrigation channel to a distal end of a catheter performing ablation. The catheter performing the ablation may also include temperature sensors (not shown) which are used to measure a temperature of the myocardium during ablation and regulate an ablation power and/or an irrigation rate of the pumping of the irrigation fluid according to the measured temperature.

Reference is now made to FIG. 3, which is a flowchart 300 including steps in a method of noise level presentation for use in the system 20 of FIG. 1.

The processing circuitry 41 is configured to receive a signal from one of the electrodes 55 (block 302), find a noise measurement of the signal (block 304), and render to the display 27 a dynamic indication of the noise measurement (block 306). In some exemplary modes, the processing circuitry is configured to receive respective signals from respective ones of the electrodes 55 (e.g., receive a signal per electrode 55), find noise measurements for the respective signals (e.g., find a noise measurement per signal), and render to the display 27 dynamic indications of the noise measurements (e.g., render a dynamic indication per signal).

The noise measurements may be computed as a function of the magnitudes of frequencies associated with noise. For example, high frequency components of the signals captured by the catheter 40 are probably of non-electro-anatomic origin. For example, frequencies above 70 or 150 Hz (or a value therebetween, depending upon the arrythmia being displayed by the patient 28) are probably of non-electro-anatomic origin and therefore noise. The noise measurements may be based on an absolute measure of the noise (e.g., magnitudes of frequencies of non-electro-anatomical origin), or as a type of signal to noise ratio (described below in more detail). In some exemplary modes, the frequency cut-off defining low and high frequencies may be set by the physician 30.

In some exemplary modes, a Fourier Transform of the signal is performed to provide magnitudes of high frequencies (e.g., noise, N) and magnitudes of low frequencies (e.g., the basic signal, S). In some exemplary modes, the magnitudes of high frequencies and low frequencies may be determined using low and/or high pass filters. A measurement of noise may be computed based on N or a type of signal-to-noise ratio may be computed as S/N or S/(S+N). The signal-to-noise ratio may be computed by software and/or hardware (e.g., circuitry).

The noise measurements are generally computed for a time window of the signals, for example, the most recent 100 milliseconds of the signal. The time window may slide as time progresses so that the computed noise measurements reflect the most recent noise of the signals. The window may have any suitable width, for example in the range of 100 milliseconds to five seconds.

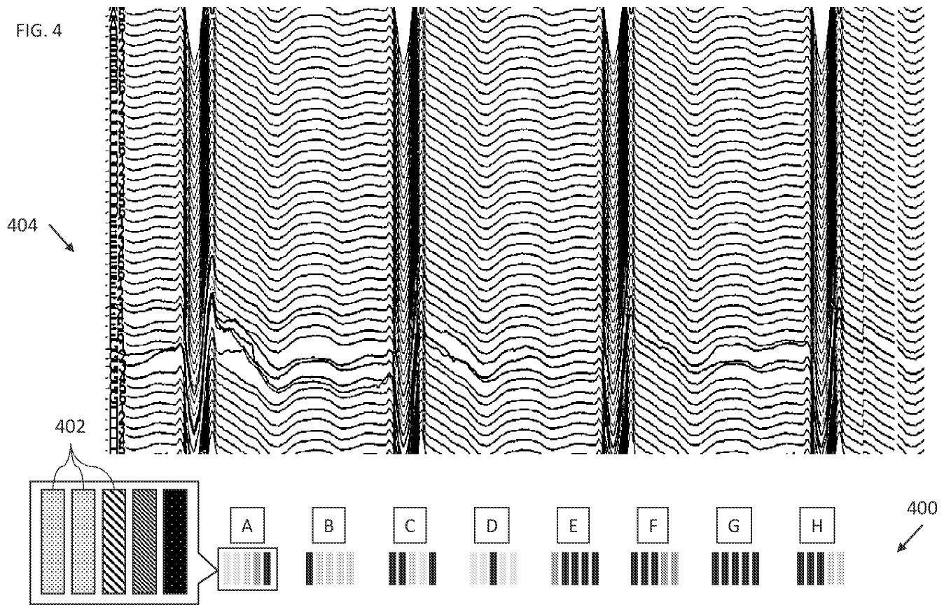

Reference is now made to FIG. 4, which is a schematic view of a noise level presentation 400 in the system 20 of FIG. 1.

FIG. 4 shows traces 404 of the IEGMs captured by the electrodes 55 of the catheter 40. The traces 404 are grouped by spline (e.g., A-H) and ordered by electrode number within each spline. In some exemplary modes, the traces 404 are not displayed with the noise level presentation 400.

The noise level presentation 400 includes dynamic indications indicating the noise measurements of the respective signals captured by the electrodes 55. In some exemplary modes, each dynamic indication is a graphical representation 402 (only some labeled for the sake of simplicity). In some exemplary modes, each graphical representation 402 includes a colored indicator which changes color responsively to a level of the respective noise measurement of that signal. In other exemplary modes, the graphical representations 402 may be shaded or patterned representations that change shading and/or patterns responsively to the level of respective noise measurement of the respective signals over time. In some exemplary modes, the graphical representations 402 may change size over time according to the level of noise, for example, a bar graph may illustrate the noise of the signals in which the respective heights of the bars change over time, and the respective heights are based on the noise measurements of the respective signals.

In some exemplary modes, each colored indicator is a colored line as shown in FIG. 4. Any suitable color scheme may be used to indicate different levels of noise. For example, “green” for no noise, “yellow” for low noise, “orange” for medium noise, and “red” for high noise. The lines change color as the noise levels of the corresponding signals change over time. The ranges of noise indicated by each color may be user configurable.

In some exemplary modes, the catheter 40 includes multiple splines 54 (e.g., eight splines associated with spline labels A-H) with the electrodes 55 being disposed along the splines 54 as shown in FIG. 2. The processing circuitry 41 is configured to render to the display 27 the graphical representations 402 grouped by the splines (e.g., A to H) as shown in FIG. 4.

The physician 30 may look at the noise level presentation 400 and decide to adjust the catheter 400 within the heart 26 to reduce noise or replace the catheter 40 if the noise level presentation 400 indicates that certain electrodes 55 are generally capturing noisy signals (independent of position of the catheter 40).

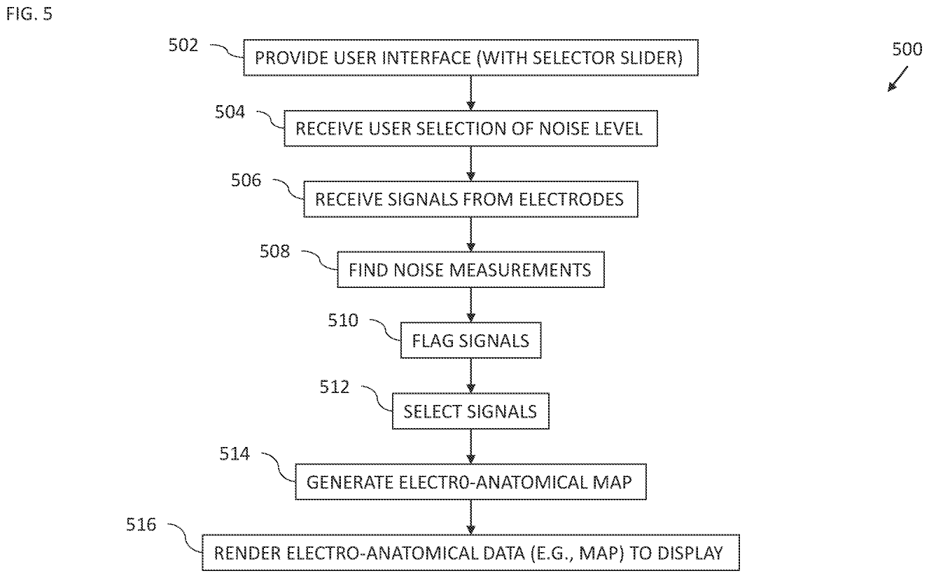

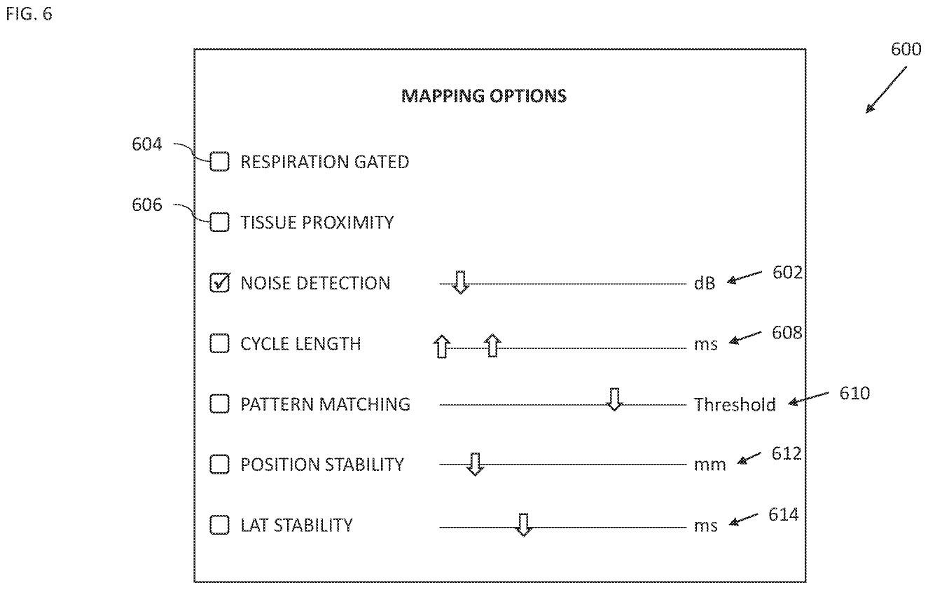

Reference is now made to FIG. 5, which is a flowchart 500 including steps in a method of setting a noise level for selecting signals in the system 20 of FIG. 1. Reference is also made to FIG. 6, which is a schematic view of a user interface 600 screen used to set a noise level for selecting signals in the system 20 of FIG. 1.

The processing circuitry 41 is configured to provide the user interface screen 600, and render the user interface screen 600 to the display 27, in order to receive a user input of a given noise level (block 502) via the user interface screen 600. In some exemplary modes, the user interface screen 600 comprises a noise level selector slider 602 to enable a user to select the given noise level by moving the slider to select the desired noise level. The processing circuitry 41 is configured to receive a user selection of the given noise level (block 504), for example, via the user adjusting the noise level selector slider 602. In some exemplary modes, the noise level may be selected by the user entering a noise level using numeric digits in the user interface screen 600, or by using keystrokes (e.g., up and down arrow keys), or foot pedal movements, or any other user interface interaction, to adjust the noise level to a desired noise level.

In some exemplary modes, the user interface screen 600 may include other mapping option selectors such as a respiration gated selector 604, a tissue proximity selector 606, a cycle length slider 608, a pattern matching slider 610, a position stability slider 612, and a LAT stability slider 614.

The catheter 40 is inserted by the physician 30 into the body part (e.g., a chamber of the heart 26) of the patient 28, as described above in more detail with reference to FIG. 1. The catheter 40 is moved around the body part and the electrodes 55 capture electrical activity from the tissue of the body part, e.g., as part of a mapping process. The processing circuitry 41 is configured to receive respective signals from respective ones of the electrodes 55 (e.g., one signal is received for each of the electrodes 55) (block 506) and find noise measurements for the respective signals (block 508). The noise measurements may be found using any suitable method, for example, using one of the methods described with reference to the step of block 304 of FIG. 3. In some exemplary modes, finding the noise measurement is performed prior to other signal processing (e.g., to reduce signal noise).

The processing circuitry 41 is configured to select signals from the received respective signals with noise measurements below the given noise level (selected by the user, e.g., the physician 30) (block 512) responsively to the found noise measurements.

In some exemplary modes, the processing circuitry 41 is configured to flag signals of the received respective signals with noise measurements below the given noise level (block 510), and select the flagged signals (block 512). In other exemplary modes, the processing circuitry 41 is configured to flag signals of the respective signals with noise measurements above or equal to the given noise level (block 510), and select non-flagged signals of the respective signals (block 512).

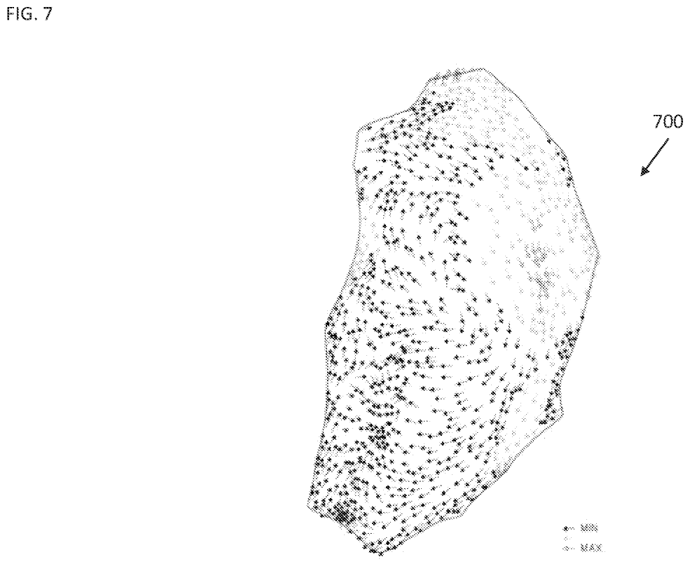

Reference is now made to FIG. 7, which is a schematic view of an electro-anatomical map 700 generated by the system 20 of FIG. 1. Reference is also made to FIG. 5. In some exemplary modes, the processing circuitry 41 is configured to generate an electro-anatomical map (such as the electro-anatomical map 700) responsively to the selected signals with noise measurements below the given noise level (block 514). In other words, signals with noise measurements below the given noise level are used to generate the electro-anatomical map 700. Any suitable electro-anatomical map may be generated, for example, showing velocity vectors (as shown in FIG. 7) or showing a LAT map or a bipolar map. The processing circuitry 41 is configured to render to the display 27 electro-anatomical data (such as the electro-anatomical map 700 and/or IEGM traces (e.g., traces 404) with low enough noise (i.e., below the given noise level) responsively to the selected signals (block 516).

In practice, some or all of the functions of the processing circuitry 41 may be combined in a single physical component or, alternatively, implemented using multiple physical components. These physical components may comprise hard-wired or programmable devices, or a combination of the two. In some examples, at least some of the functions of the processing circuitry 41 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 72% to 108%.

### EXAMPLES

Example 1: A medical system, comprising: a catheter configured to be inserted into a body part of a living subject, and comprising multiple electrodes configured to contact tissue of the body part; a display; and processing circuitry configured to: receive a signal from one of the electrodes; find a noise measurement of the signal; and render to the display a dynamic indication of the noise measurement.

Example 2: The system according to example 1, wherein the dynamic indication is a graphical representation.

Example 3: The system according to example 2, wherein the graphical representation includes a colored indicator which changes color responsively to a level of the noise measurement.

Example 4: The system according to example 3, wherein the colored indicator is a colored line.

Example 5: The system according to any of examples 1-4, wherein the processing circuitry is configured to: receive respective signals from respective ones of the electrodes; find noise measurements for the respective signals; and render to the display dynamic indications of the noise measurements.

Example 6: The system according to example 5, wherein the dynamic indications are graphical representations.

Example 7: The system according to example 6, wherein the graphical representations include colored indicators which change color responsively to respective levels of the noise measurements.

Example 8: The system according to example 7, wherein the colored indicators are colored lines.

Example 9: The system according to any of examples 1-8, wherein: the catheter includes multiple splines with the electrodes being disposed among the splines; and the processing circuitry is configured to render to the display the graphical representations grouped by the splines.

Example 10: The system according to any of examples 1-9, wherein the processing circuitry is configured to: receive respective signals from respective ones of the electrodes; find noise measurements for the respective signals; select signals from the respective signals with noise measurements below a given noise level; and render to the display electro-anatomical data responsively to the selected signals.

Example 11: The system according to example 10, wherein the processing circuitry is configured to: generate an electro-anatomical map responsively to the selected signals with noise measurements below the given noise level; and render the electro-anatomical map to the display.

Example 12: The system according to example 10 or 11, wherein the processing circuitry is configured to provide a user interface screen to receive a user input of the given noise level.

Example 13: The system according to example 12, wherein the user interface screen comprises a noise level selector slider to enable a user to select the given noise level.

Example 14: The system according to any of examples 10-13, wherein the processing circuitry is configured to: flag signals of the respective signals with noise measurements below the given noise level; and select the flagged signals.

Example 15: The system according to any of examples 10-13, wherein the processing circuitry is configured to: flag signals of the respective signals with noise measurements above or equal to the given noise level; and select non-flagged signals of the respective signals.

Example 16: A medical method, comprising: receiving a signal from an electrode of a catheter, the electrode being configured to contact tissue of a body part of a living subject; finding a noise measurement of the signal; and rendering to a display a dynamic indication of the noise measurement.

Example 17: A software product, comprising a non-transient computer-readable medium in which program instructions are stored, which instructions, when read by a central processing unit (CPU), cause the CPU to: receive a signal from an electrode of a catheter, the electrode being configured to contact tissue of a body part of a living subject; find a noise measurement of the signal; and render to a display a dynamic indication of the noise measurement.

Example 18: A medical system, comprising: a catheter configured to be inserted into a body part of a living subject, and comprising multiple electrodes configured to contact tissue of the body part; a display; and processing circuitry configured to: receive respective signals from respective ones of the electrodes; find noise measurements for the respective signals; select signals from the respective signals with noise measurements below a given noise level; and render to the display electro-anatomical data responsively to the selected signals.

Example 19: The system according to example 18, wherein the processing circuitry is configured to: generate an electro-anatomical map responsively to the selected signals with noise measurements below the given noise level; and render the electro-anatomical map to the display.

Example 20: The system according to example 18 or 19, wherein the processing circuitry is configured to provide a user interface screen to receive a user input of the given noise level.

Example 21: The system according to example 20, wherein the user interface screen comprises a noise level selector slider to enable a user to select the given noise level.

Example 22: The system according to any of examples 18-21, wherein the processing circuitry is configured to: flag signals of the respective signals with noise measurements below the given noise level; and select the flagged signals.

Example 23: The system according to any of examples 18-21, wherein the processing circuitry is configured to: flag signals of the respective signals with noise measurements above or equal to the given noise level; and select non-flagged signals of the respective signals.

Example 24: A medical method, comprising: receiving respective signals from respective electrodes of a catheter inserted into a body part of a living subject; finding noise measurements for the respective signals; selecting signals from the respective signals with noise measurements below a given noise level; and rendering to a display electro-anatomical data responsively to the selected signals.

Example 25: A software product, comprising a non-transient computer-readable medium in which program instructions are stored, which instructions, when read by a central processing unit (CPU), cause the CPU to: receive respective signals from respective electrodes of a catheter inserted into a body part of a living subject; find noise measurements for the respective signals; select signals from the respective signals with noise measurements below a given noise level; and render to a display electro-anatomical data responsively to the selected signals.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.

The examples described above are cited by way of example, and the present disclosure is not limited by what has been particularly shown and described hereinabove. Rather the scope of the disclosure includes both combinations and sub-combinations of the various features described hereinabove, as well as variations and modifications thereof which would occur to persons skilled in the art upon reading the foregoing description and which are not disclosed in the prior art.

What is claimed is:

1. A medical system, comprising:
a catheter configured to be inserted into a body part of a living subject, and comprising multiple electrodes configured to contact tissue of the body part;
a display; and
processing circuitry configured to:
receive a signal from one of the electrodes;
find a noise measurement of the signal; and
render to the display a dynamic indication of the noise measurement.

2. The system according to claim 1, wherein the dynamic indication is a graphical representation.

3. The system according to claim 2, wherein the graphical representation includes a colored indicator which changes color responsively to a level of the noise measurement.

4. The system according to claim 3, wherein the colored indicator is a colored line.

5. The system according to claim 1, wherein the processing circuitry is configured to:
receive respective signals from respective ones of the electrodes;
find noise measurements for the respective signals; and
render to the display dynamic indications of the noise measurements.

6. The system according to claim 5, wherein the dynamic indications are graphical representations.

7. The system according to claim 6, wherein the graphical representations include colored indicators which change color responsively to respective levels of the noise measurements.

8. The system according to claim 7, wherein the colored indicators are colored lines.

9. The system according to claim 6, wherein:
the catheter includes multiple splines with the electrodes being disposed among the splines; and
the processing circuitry is configured to render to the display the graphical representations grouped by the splines.

10. The system according to claim 1, wherein the processing circuitry is configured to:
receive respective signals from respective ones of the electrodes;
find noise measurements for the respective signals;
select signals from the respective signals with noise measurements below a given noise level; and
render to the display electro-anatomical data responsively to the selected signals.

11. The system according to claim 10, wherein the processing circuitry is configured to:
generate an electro-anatomical map responsively to the selected signals with noise measurements below the given noise level; and
render the electro-anatomical map to the display.

12. The system according to claim 10, wherein the processing circuitry is configured to provide a user interface screen to receive a user input of the given noise level.

13. The system according to claim 12, wherein the user interface screen comprises a noise level selector slider to enable a user to select the given noise level.

14. The system according to claim 10, wherein the processing circuitry is configured to:
flag signals of the respective signals with noise measurements below the given noise level; and
select the flagged signals.

15. The system according to claim 10, wherein the processing circuitry is configured to:
flag signals of the respective signals with noise measurements above or equal to the given noise level; and
select non-flagged signals of the respective signals.

16. A medical method, comprising:
receiving a signal from an electrode of a catheter, the electrode being configured to contact tissue of a body part of a living subject;
finding a noise measurement of the signal; and
rendering to a display a dynamic indication of the noise measurement.

17. A software product, comprising a non-transient computer-readable medium in which program instructions are stored, which instructions, when read by a central processing unit (CPU), cause the CPU to:
receive a signal from an electrode of a catheter, the electrode being configured to contact tissue of a body part of a living subject;
find a noise measurement of the signal; and
render to a display a dynamic indication of the noise measurement.

18. A medical system, comprising:
a catheter configured to be inserted into a body part of a living subject, and comprising multiple electrodes configured to contact tissue of the body part;
a display; and
processing circuitry configured to:
receive respective signals from respective ones of the electrodes;
find noise measurements for the respective signals;
select signals from the respective signals with noise measurements below a given noise level; and
render to the display electro-anatomical data responsively to the selected signals.

19. The system according to claim 18, wherein the processing circuitry is configured to:
generate an electro-anatomical map responsively to the selected signals with noise measurements below the given noise level; and
render the electro-anatomical map to the display.

20. The system according to claim 18, wherein the processing circuitry is configured to provide a user interface screen to receive a user input of the given noise level.

21. The system according to claim 20, wherein the user interface screen comprises a noise level selector slider to enable a user to select the given noise level.

22. The system according to claim 18, wherein the processing circuitry is configured to:
flag signals of the respective signals with noise measurements below the given noise level; and
select the flagged signals.

23. The system according to claim 18, wherein the processing circuitry is configured to:
flag signals of the respective signals with noise measurements above or equal to the given noise level; and
select non-flagged signals of the respective signals.

24. A medical method, comprising:
receiving respective signals from respective electrodes of a catheter inserted into a body part of a living subject;
finding noise measurements for the respective signals;
selecting signals from the respective signals with noise measurements below a given noise level; and
rendering to a display electro-anatomical data responsively to the selected signals.

25. A software product, comprising a non-transient computer-readable medium in which program instructions are stored, which instructions, when read by a central processing unit (CPU), cause the CPU to:
receive respective signals from respective electrodes of a catheter inserted into a body part of a living subject;
find noise measurements for the respective signals;
select signals from the respective signals with noise measurements below a given noise level; and
render to a display electro-anatomical data responsively to the selected signals.
